(19) United States
(12) Patent Application Publication
(10) Pub. No.: US 2005/0159666 A1
(43) Pub. Date: Jul. 21, 2005

(54) Apparatus and methods for documenting myocardial ischemia

(76) Inventors: Christopher Pearce, Monroe, WA (US); David Hampton, Woodinville, WA (US)

Correspondence Address: INGRASSIA FISHER & LORENZ, P.C., 7150 E. CAMELBACK, STE. 325, SCOTTSDALE, AZ, 85251, US

(21) Appl. No.: 10/762,387

(22) Filed: Jan. 21, 2004

(51) Int. Cl. A61B 5/0402

(52) U.S. Cl. 600/509

(57) ABSTRACT

Methods and apparatus are provided for documenting the myocardial ischemia of a patient's heart. The apparatus comprises an ECG monitor and data collector configured to receive electrocardial data about the patient's heart. The apparatus further comprises a cardiac marker data collector configured to receive cardiac marker data about the patient's heart. A data processing and recording module is in electrical communication with the ECG monitor and data collector and the cardiac marker data collector and is configured to record the electrocardial data and the cardiac marker data. The method comprises the steps of obtaining electrocardial data about the patient's heart and receiving results of a cardiac marker test performed on the patient. The electrocardial data and the test results are stored in a patient report, which then may be displayed.

### FIELD OF THE INVENTION

The present invention generally relates to medical devices for monitoring cardiac episodes, and more particularly relates to devices and techniques for documenting the presence of myocardial ischemia in patients.

### BACKGROUND OF THE INVENTION

Myocardial ischemia is the result of an imbalance between myocardial oxygen supply and demand and is a reversible process if blood flow is restored before cellular damage occurs. Ischemia can result from increased myocardial oxygen demands or from decreased myocardial oxygen supply. If ischemia is severe and blood flow is not restored relatively soon, cellular injury and eventually necrosis (infarction) result. Myocardial infarction can occur because of blockage of a coronary artery with thrombus or from severe and prolonged ischemia due to coronary artery spasm or unrelieved obstruction of a coronary artery.

Angina pectoris (angina), literally “strangling of the chest,” is often a symptom of myocardial ischemia. Angina is traditionally classified as “stable” or “unstable”, depending upon severity. Stable angina, the least alarming form of ischemia, is caused by a high demand for oxygen by the myocardium during exertion. It is characterized by transient episodes of substemal chest pain or discomfort and is usually relieved by rest. Unstable angina, which occurs when the patient is at rest or is not relieved by rest, may be classified as intermediate in severity between stable angina and myocardial infarction.

Electrocardiography (ECG) is a cost-effective test that is widely available for the diagnosis of myocardial ischemia in patients. Unequivocal ECG changes that indicate myocardial infarction include the development of abnormal, persistent Q waves, the presence of a QS complex in two or more leads, or an evolving injury current pattern lasting longer than one day. The presence of Q waves indicates infarcted tissue that extends at least halfway through the myocardial wall.

However, ECG results do not always definitively indicate the presence or absence of acute ischemia. For example, common patterns of ischemia include T-wave inversion and ST segment elevation, although T-wave inversion and ST segment elevation are often non-specific findings and can be due to a variety of causes other than ischemia, such as cardiomyopathies, pulmonary embolism, pericarditis, and subarachnoid hemorrhage. Other equivocal ECG changes that are not diagnostic but are suggestive of myocardial infarction consist of an abnormal Q wave or conduction disturbances. In addition, the diagnosis of acute myocardial infarction becomes difficult or impossible when there are preexisting ECG abnormalities such as left bundle-branch block, an old myocardial infarction in the same area, ventricular hypertrophy, and Wolff-Parkinson-White syndrome.

To confirm the presence of myocardial infarction suggested by ECG results, other tests may be performed on the patient. For example, certain serum markers rise in a patient after a myocardial infarction. Creatine kinase (CK) and its iso-enzyme MB (CK-MB) are enzymes released with tissue necrosis. Myoglobin, a heme protein, is found in all striated tissue and is released from myocytes after tissue injury. Troponin T or troponin I is released upon cardiac necrosis. Thus, cardiac marker tests of a patient's blood to determine the presence and levels of serum markers may facilitate confirmation of myocardial infarction.

However, the various tests conducted to confirm myocardial ischemia may be performed by a number of different technicians or other emergency personnel using various medical equipment at various locations. Accordingly, compilation of the information necessary for a physician, a clinician or other medical personnel to make an accurate and timely diagnosis of myocardial ischemia may be difficult. Further, during an emergency response situation, when a patient is experiencing painful angina, the performance of certain tests that may facilitate diagnosis may be overlooked or may be performed inaccurately.

Accordingly, it is desirable to provide an apparatus for documenting and storing in one location various monitoring and test results that may indicate myocardial ischemia in a patient's heart. In addition, it is desirable to provide a method for documenting in one location myocardial ischemia in a patient's heart. Furthermore, other desirable features and characteristics of the present invention will become apparent from the subsequent detailed description of the invention and the appended claims, taken in conjunction with the accompanying drawings and this background of the invention.

### BRIEF SUMMARY OF THE INVENTION

In accordance with an exemplary embodiment of the present invention, there is provided an apparatus for documenting the myocardial ischemia of a patient's heart. The apparatus comprises an ECG monitor and data collector configured to receive electrocardial data about the patient's heart. The apparatus further comprises a cardiac marker data collector configured to receive cardiac marker data about the patient's heart. A data processing and recording module is in electrical communication with the ECG monitor and data collector and the cardiac marker data collector and is configured to record the electrocardial data and the cardiac marker data.

In accordance with another exemplary embodiment of the invention, there is provided a method for documenting the myocardial ischemia of a patient's heart. The method comprises the steps of obtaining electrocardial data about the patient's heart and receiving results of a cardiac marker test performed on the patient. The electrocardial data and the test results are stored in a patient report, which then may be displayed.

In accordance with a further exemplary embodiment of the invention, there is provided an apparatus for documenting the myocardial ischemia of a patient's heart. The apparatus comprises means for receiving electrocardial data about the patient's heart and means for receiving cardiac marker data about the patient's heart. The apparatus further comprises a means for processing the electrocardial data and the cardiac marker data into a patient record and means for displaying the patient record.

In yet another exemplary embodiment of the invention, there is provided a medical apparatus of the type that is configured to monitor the electrocardiogram waveform of a patient. The medical apparatus comprises a cardiac marker data collector configured to receive cardiac marker data about the patient's heart. The medical apparatus also comprises a data processor in electrical communication with the cardiac marker data collector. A memory module is in electrical communication with the data processor and is configured to record the electrocardiogram waveform and the cardiac marker data. A display module is in electrical communication with the data processor and is configured to display at least one of the electrocardiogram waveform and the cardiac marker data.

### BRIEF DESCRIPTION OF THE DRAWINGS

The present invention will hereinafter be described in conjunction with the following drawing figures, wherein like numerals denote like elements, and

FIG. 1 is a simplified schematic block diagram of an apparatus for documenting myocardial ischemia of a patient's heart in accordance with an exemplary embodiment of the present invention;

FIG. 2 is schematic illustration of an apparatus for documenting myocardial ischemia in accordance with an exemplary embodiment of the present invention; and

FIG. 3 is a simplified flow chart of a process for documenting myocardial ischemia of a patient's heart in accordance with an exemplary embodiment of the present invention.

### DETAILED DESCRIPTION OF THE INVENTION

The following detailed description of the invention is merely exemplary in nature and is not intended to limit the invention or the application and uses of the invention. Furthermore, there is no intention to be bound by any theory presented in the preceding background of the invention or the following detailed description of the invention.

According to various embodiments of the present invention, an apparatus is configured to document the myocardial ischemia of a patient's heart. The apparatus monitors and collects data regarding the electrocardiogram of the patient's heart and also collects data regarding the results of a cardiac marker test performed on the patient. In other embodiments of the invention, the apparatus also may monitor various other parameters of the patient's condition and gather data regarding the patient and the patient's cardiac state. The apparatus then may display the various data together to facilitate the identification and/or treatment of myocardial ischemia in the patient's heart. In this manner, data that will facilitate the accurate and timely diagnosis of myocardial ischemia may be generated, collected, and stored in one apparatus and may be presented to a physician, clinician, technician or the like in a comprehensive patient report.

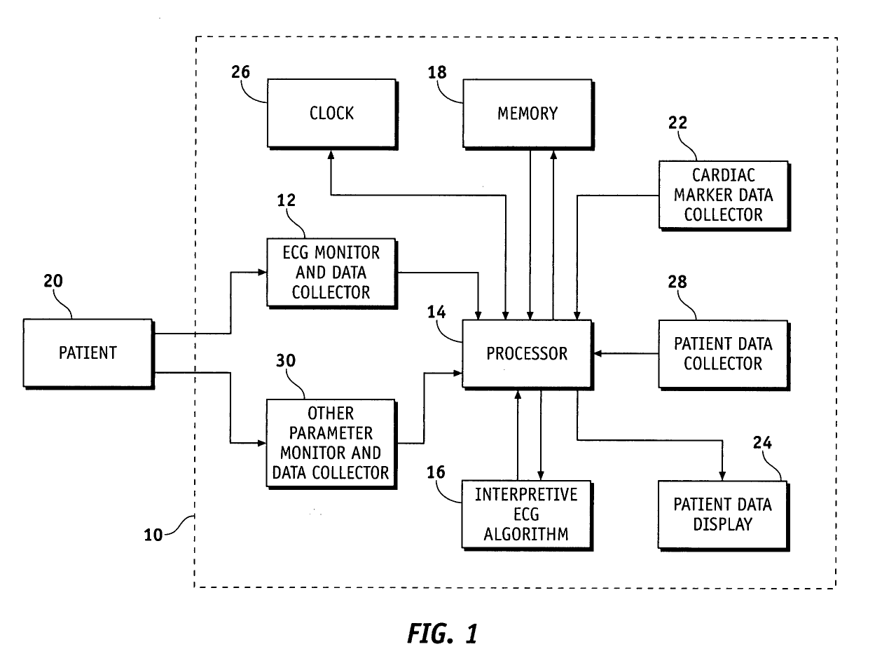

FIG. 1 is a simplified schematic block diagram of an apparatus 10 for documenting the myocardial ischemia of a patient's heart, in accordance with one exemplary embodiment of the present invention. Apparatus 10 suitably comprises an ECG monitor and data collector module 12, a cardiac marker data collector module 22, a processor module 14, and a patient data display module 24. Each of the various modules may be implemented with computer-executable instructions stored in a memory 18 and executed on processor module 14, or in any other manner.

ECG monitor and data collector module 12 is any circuit, programming routine, application and/or other hardware/software that permits suitable coupling of apparatus 10 to a patient 20 and that generates electrical impulses or other observed signals that can be used to model the patient's electrocardiogram (“PQRST”) waveform (hereinafter “ECG data”). ECG monitor and data collector module 12 is in electrical communication with processor 14 and is configured to transmit the ECG data to processor 14 for analysis and/or storage. ECG monitor and data collector module 12 may utilize any suitable ECG monitoring configuration but preferably utilizes an industry standard 12-lead configuration. ECG monitor and data collector module 12 may be coupled to patient 20 for electrocardiogram monitoring using any suitable number of sensor connections, such as 4-wire connections, 6-wire connections, 10-wire connections and the like. In addition to the ECG data, ECG monitor and data collector module 12 may be configured to gather data regarding the time and date of the ECG monitoring of a patient.

In a further exemplary embodiment of the invention, apparatus 10 may interpret the data collected by ECG monitor and data collector module 12 to indicate or suggest to a user of apparatus 10, or to a physician, or other clinician or technician, that myocardial ischemia of the heart of patient 20 is or may be present. In this embodiment of the invention, apparatus 10 may comprise an interpretive ECG algorithm module 16, which may be any circuit, programming routine and/or hardware/software that receives ECG data from processor 14, analyzes the data, and sends an interpretation of the data back to processor 14 for delivery to a user or other person. Interpretive ECG algorithm 16 may utilize any suitable algorithm that analyzes the ECG data collected by ECG monitor and data collector 12 and interprets the data to determine if myocardial ischemia is suggested by the data. Preferably, interpretive ECG algorithm 16 comprises an industry-standard interpretive 12-lead algorithm. Examples of suitable algorithms include the GE Marquette 12SL ECG analysis program manufactured by GE Medical Systems of Waukesha, Wis. and the Glasgow Royal Infirmary Interpretive ECG algorithm developed by the University of Glasgow of Glasgow, Scotland.

Processor 14 is any circuit, programming routine, application or other hardware/software module that is capable of processing data received from ECG monitor and data collector 12, and any of the other various patient parameter monitors described below, and causing such data to be displayed. Processor 14 may be implemented with any type of microprocessor, digital signal processor, application specific integrated circuit (ASIC) or other integrated or discrete logic circuitry programmed or otherwise configured to provide functionality as described herein. Processor 14 executes instructions stored in a digital memory 18 to provide functionality as described below. Instructions provided to processor 14 may be executed in any manner, using any data structures, architecture, programming language and/or other techniques. Digital memory 18 is any storage medium capable of maintaining digital data and instructions provided to processor 14, such as a static or dynamic random access memory (RAM), or any other electronic, magnetic, optical or other storage medium.

Apparatus 10 further comprises a cardiac marker data collector module 22, which is any circuit, programming routine, application or other hardware/software module that is configured to receive data regarding the results of a cardiac marker test performed on the patient. Cardiac marker data collector module 22 may be coupled to processor 14 and is configured to transmit cardiac marker test results data to processor 14. Processor 14 then may transmit that data to memory 18 for storage and later retrieval or may transmit the data for display, as described in more detail below. Data may be received by cardiac marker data collector in any suitable form. For example, data may be received as a “positive” or “negative” election, that is, the data may indicate results positively confirming cardiac marker elevation or may indicate no cardiac maker elevation. Alternatively, the data may be received as a numeric indication of the level of a cardiac marker(s) in the patient's blood. Accordingly, any suitable cardiac marker test may be performed on patient 20 and the results may be entered using cardiac marker data collector 22. An example of a cardiac marker test suitable for use with the present invention includes, but is not limited to, the Cardiac Status™ point-of-care test kit manufactured by Spectral Diagnostics, Inc. of Toronto, Ontario, Canada. Cardiac marker data collector 22 may comprise any suitable data input configuration, such as, for example, a keypad or touch screen, used for entering cardiac marker test results into cardiac marker data collector 22. In various embodiments of the invention, cardiac marker data collector 22 also may be configured to identify the time and date of entry of the cardiac marker test results. In this manner, later confirmation that a cardiac marker test was performed in accordance with a prescribed emergency protocol or routine may be confirmed.

In one exemplary embodiment of the invention, processor 14 or other suitable component of apparatus 10 may be configured to interpret the ECG data collected by ECG monitor and collector module 12 in conjunction with cardiac marker test results collected by cardiac marker data collector 22 to suggest or indicate to a user of apparatus 10, or to a physician or other clinician or technician, that myocardial ischemia is or may be present in the heart of patient 20. In this manner, if interpretive ECG algorithm 16 is not able to confirm the presence of myocardial ischemia, processor 14 may enhance the certainty with which apparatus 10 may diagnose myocardial ischemia by assessing both the ECG data, and/or the interpretation of the ECG data by interpretive ECG algorithm 16, and the results of the cardiac marker test. Once processor 14 has assessed the data, processor's 14 interpretation of the data may be displayed to the user of apparatus 10 or any other physician, clinician or technician via a patient data display module 24, described in more detail below. In another embodiment of the present invention, processor 14 may cause to be displayed a suggested course of treatment or other action that is based on the diagnosis.

Apparatus 10 also comprises a patient data display module 24, which comprises any circuit, programming routine, application or hardware/software module that suitably displays data received from processor 14, such as data collected by ECG monitor and collector module 12 and cardiac marker data collector 22. Patient data display 24 may comprise any display suitable for providing to a user, technician, physician, clinician, or the like the ECG data gathered by ECG monitor and data collector 12 and the cardiac marker test results gathered by cardiac marker data collector 22. In one exemplary embodiment of the invention, patient data display 24 may comprise a visual display, such as a liquid crystal display, a CRT screen, a television screen, and the like. In another exemplary embodiment of the invention, patient data display 24 may comprise a printer that prints a patient report of any of the data received from processor 14. In a further exemplary embodiment of the invention, patient data display 24 may comprise both a visual display and printer. In this manner, ECG data and cardiac marker tests results may be displayed via a visual display to the user of apparatus 10 and/or also may be stored in memory 18 for later display in a printed patient report.

In another exemplary embodiment of the present invention, apparatus 10 may be configured to provide instructions to a user to conduct a cardiac marker test depending on the results of the ECG data collected by ECG monitor and data collector 12 and interpreted by interpretive ECG algorithm 16. For example, if interpretive ECG algorithm 16 interprets the ECG data and concludes that the ECG data may suggest myocardial ischemia but that such a determination is not definitive, processor 14, which receives and processes data received from interpretive ECG algorithm 16, may produce a prompt instructing the user to perform a cardiac marker test. The prompt may be provided as instructions appearing on a visual display, such as patient data display 24, as a signal light appearing on apparatus 10, or may be an alarm or other auditory signal, or any other suitable prompt or signal. Alternatively, if interpretive ECG algorithm 16 interprets the ECG data and concludes that the ECG data indicates myocardial ischemia, processor 14 may be configured so that a user is not provided a prompt to conduct a cardiac marker test. In another embodiment of the invention, apparatus 10 may be configured to provide instructions to a user to conduct a cardiac marker test regardless of the ECG data or any interpretation thereof.

In yet another exemplary embodiment of the present invention, apparatus 10 may provide a prompt to the user to enter cardiac marker test results into cardiac marker data collector 22 after a predetermined amount of time has elapsed from initiation of the cardiac marker test. In this embodiment of the invention, apparatus 10 may provide a prompt to a user to conduct a cardiac marker test, as described above. The user then may be required to indicate, via a keypad, a touch screen or other suitable device of apparatus 10, the commencement of a cardiac marker test. Processor 14 receives data indicating the commencement of a cardiac marker test and initiates operation of a clock 26 or other timing device. Processor 14 then monitors clock 26 to determine if a preset period of time has elapsed. When a preset period of time has elapsed, processor 14 may provide an alarm, a visual prompt, or other suitable notification to the user to enter the cardiac marker test results into cardiac marker data collector 22. Apparatus 10 may monitor a preprogrammed period of time for performance of a cardiac marker test or, alternatively, apparatus 10 may be configured to permit a user to program a desired time period for performance of a cardiac marker test.

In another exemplary embodiment of the invention, processor 14 of apparatus 10 may be configured to monitor and compute the change over time of the electrocardiogram waveform of patient 20 and/or the circulating levels of the cardiac markers in patient 20. In this manner, a determination of mutually reinforcing changes in the electrocardiogram waveform of patient 20 and the circulating levels of the cardiac markers in patient 20 may be used to further enhance diagnostic accuracy and to evaluate the effectiveness of any therapies being provided to patient 20. For example, if processor 14 detects a change in the electrocardiogram over a preset period of time, apparatus 10 may prompt the user to perform a cardiac marker test. If processor 14 determines that the results of the cardiac marker test confirm the presence or absence of myocardial ischemia, processor 14 may cause the diagnosis to be displayed, such as via patient data display module 24. Processor 14 also may cause a suggested treatment to be displayed. In another example, if processor 14 determines that concurrent changes in the electrocardiogram waveform and in the cardiac marker test results indicate that myocardial infarction is imminent, it may cause this diagnosis to be displayed and/or cause a suggested treatment to be displayed.

Apparatus 10 further may comprise a patient data collector 28, which is operably connected to processor 14. Patient data collector 28 may receive identifying information of the patient and other data that may facilitate the diagnosis of myocardial ischemia of the patient's heart. Examples of patient data that may be received by patient data collector 28 include the patient's name and/or an identification number associated with the patient, the patient's age, the patient's sex, the patient's race, and the like. Patient data collector 28 may utilize any suitable data input configuration, such as, for example, a keypad, keyboard, or touch screen, used for entering patient data.

Apparatus 10 also may comprise one or more monitors and collectors 30 of other physiological parameters of the patient that may facilitate the diagnosis of myocardial ischemia in patient 20. Monitor and collector(s) 30 may be suitably coupled to patient 20 to monitor and gather data relating to the physiological state of patient 20 and may transmit the data to processor 14. Processor 14 then may cause the data to be stored in memory 18 and/or may cause the data to be displayed via patient data display 24. Monitor and collector(s) 30 may be suitably coupled to patient 20 to monitor and collect data regarding any suitable parameter indicative of the physiological state of patient 20, such as, for example, the heart rate of patient 20, the oxygen saturation of the patient's 20 hemoglobin, the venous and/or arterial blood pressure of patient 20, and/or the end-tidal carbon dioxide of patient 20. Apparatus 10 may suitably display the data gathered by one monitor and collector 30 or, alternatively may display the data gathered by two or more monitors and collectors 30.

It will be appreciated that the exemplary blocks shown in FIG. 1 are intended to illustrate one logical model for implementing an apparatus for documenting the myocardial ischemia of a patient's heart. However, the model illustrated in FIG. 1 should not be construed as limiting. Indeed, the various practical embodiments may have widely varying software modules, data structures, applications, processes and the like. As such, the various functions of each block or module of FIG. 1 may in practice be combined, augmented, optimized or otherwise differently organized in any fashion.

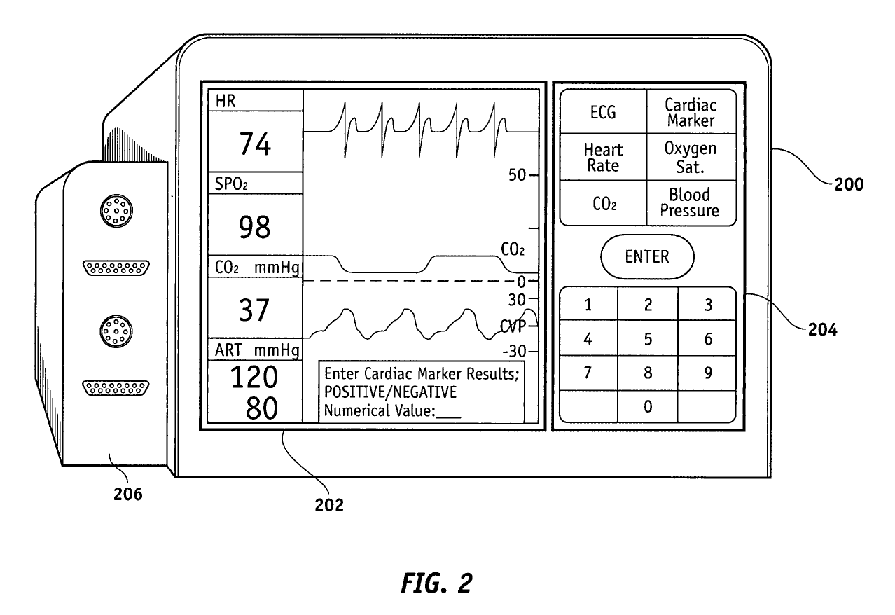

The various above-described components of apparatus 10 may be housed in a common housing 200, such as that illustrated in FIG. 2. Housing 200 may comprise patient data display module 24 (FIG. 1) that utilizes a visual display 202. Visual display 202 may be configured to display the data collected by ECG monitor and collection module 22 and cardiac marker data collector 22 (FIG. 1), the patient data collected by patient data collector 28 (FIG. 1), and/or the patient physiological parameters collected by monitor and collector module(s) 30. Housing 200 also may comprise a user interface 204, such as, for example, a keyboard, keypad, or a touch screen, that is configured to receive from a user of apparatus 10 instructions or data, such as, for example, cardiac marker test results and/or patient identification data. Housing 200 further may comprise connectors 206 that are configured to receive sensor connections (not shown), that suitably couple a patient to apparatus 10, such as ECG lead wires, blood pressure monitors, carbon dioxide monitors, and the like.

In an alternative embodiment of the invention, portions of apparatus 10 may be housed separately from housing 200. For example, where patient data display 24 comprises a printer for printing out a patient report, the printer could be integrated with apparatus 10 or provided in a separate housing. In this case, processor 14 housed within housing 200 may interact with the printer via an electrical cable or wireless link. Alternatively, apparatus 10 may comprise a printer disposed at least partially within housing 200 and a printer disposed remotely from housing 200. In another example, a visual display may be located remotely from housing 200, such as in a hospital or doctor's office, and may interact with processor 14 via an electrical cable or wireless link. Alternatively, apparatus 10 may comprise a visual display disposed within or as part of housing 200 and a visual display disposed remotely from housing 200.

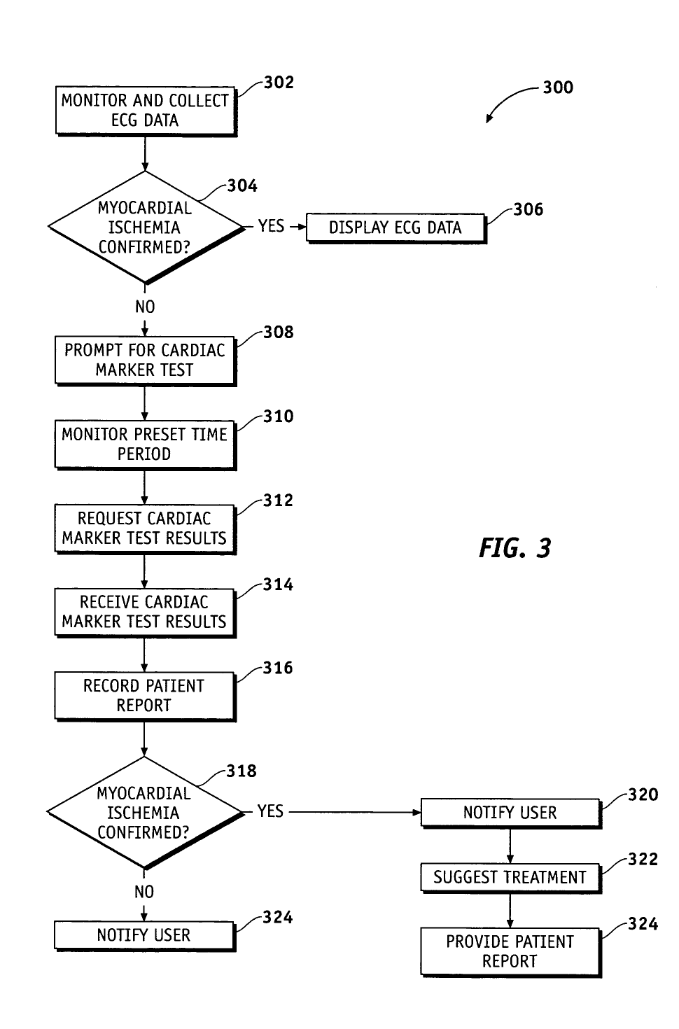

With reference now to FIG. 3, an exemplary process 300 for documenting myocardial ischemia of a patient's heart generally includes the steps of monitoring and collecting ECG data (step 302), receiving cardiac marker test results (step 314) and displaying a patient report (step 322). In an exemplary embodiment of the present invention, the various steps of process 300 may be implemented with computer-executable instructions that are stored in digital memory 18 and that are appropriately executed by processor 14 (FIG. 1), or by any other processor associated with apparatus 10.

Process 300 suitably begins by monitoring and collecting ECG data (step 302) of a patient when a user of apparatus 10 initiates ECG monitoring. A user may initiate ECG monitoring in any suitable manner, such as by selecting or activating an ECG function key of user interface 204 (FIG. 2), or by coupling the patient to apparatus 10 using suitable ECG sensor lead wires. Process 300 also may include the monitoring and collecting of data regarding other patient physiological parameters. As described above, such patient parameters may include, but are not limited to, the patient's heart rate, the oxygen saturation of the patient's hemoglobin, the arterial and/or venous blood pressure of the patient, and the end-tidal carbon dioxide of the patient. Data may be collected according to any scheme, but in an exemplary embodiment data measurements are taken at regular intervals with a sufficiently high frequency so that the data may facilitate diagnosis of myocardial ischemia (e.g., on the order of every few minutes or seconds). After the data is obtained, it is formatted or otherwise processed as appropriate to put the data into a format that can be readily received and processed by processor 14 (FIG. 1) or another appropriate component of apparatus 10.

Upon receipt of the ECG data, processor 14 may cause the ECG data to be stored in memory 18 and/or displayed, such as on patient data display 24. Alternatively, in the embodiment shown in FIG. 3, the ECG data collected by ECG monitor and data collector module 12 is suitably processed and interpreted to determine if it is suggestive of myocardial ischemia (step 304). If myocardial ischemia is determined to be present in the patient's heart, in one exemplary embodiment of the invention, the ECG data may be displayed in a patient report, such as on visual display 202 (FIG. 2) or in a printed patient report (step 306). In another exemplary embodiment of the invention, a notification that myocardial ischemia is detected may also be displayed. In a further exemplary embodiment of the invention, the severity of the myocardial ischemia may be analyzed by apparatus 10 and a suggested treatment based on the determined severity may be displayed.

In one embodiment of the invention, if myocardial ischemia is suggested by the ECG data but cannot be definitively determined, apparatus 10 may issue a prompt for the performance of a cardiac marker test (step 308). In an alternative embodiment of the invention, apparatus 10 may issue a prompt for the performance of a cardiac marker test regardless of the ECG data. The prompt may be issued in any suitable manner, such as via an audible signal to the user or on visual display 202 (FIG. 2).

In another, optional, embodiment of the invention, apparatus 10 may monitor a preset time period during which a cardiac marker test is to be conducted (step 310). The preset time period may be programmed by the user of apparatus 10 or, alternatively, may be a pre-programmed time period. Apparatus 10 may begin monitoring the preset time period upon issuing instructions to a user to conduct a cardiac marker test or, alternatively, may begin monitoring the preset time period upon activation by the user of a key, such as a cardiac marker function key, button or other device that signals to apparatus 10 that the user is commencing a cardiac marker test. Apparatus 10 may monitor the preset time period using any suitable method or device, such as a clock or timer.

Apparatus 10 then may request a user to enter the results of the cardiac marker test (step 312). Apparatus 10 may request the results after it has determined that the preset time period described above has passed or, alternatively, may request the results upon selection or activation by a user of a cardiac marker function key of user interface 204. The results may be entered using any suitable data input device or mechanism, such as, for example, a keypad or touch screen, of user interface 204 used for entering cardiac marker test results into cardiac marker data collector 22. The results of the cardiac marker test are received by cardiac marker data collector 22 (step 314), and the data is formatted or otherwise processed as appropriate to put the data into a format that can be readily received and processed by processor 14 or another appropriate component of apparatus 10. The data then may be transferred by processor 14 or another appropriate component of apparatus 10 to memory 18 for later incorporation into a patient report or to patient data display 24. In addition to the cardiac marker test results, apparatus 10 may record the date and time that the cardiac marker test results were received.

In another exemplary embodiment of the invention, processor 14 or another appropriate component of apparatus 10 may process the ECG data and the results of the cardiac marker test to determine if myocardial ischemia is present (step 318). If apparatus 10 concludes that no myocardial ischemia is present, apparatus 10 may so notify the user (step 324) or, alternatively, may instruct the user to continue with monitoring of the patient's ECG waveform and/or conduct an additional cardiac marker test. If apparatus 10 concludes that myocardial ischemia is present, apparatus 10 may so notify the user (step 320). In another embodiment of the invention, apparatus 10 may notify the user of the indication of myocardial ischemia and may suggest a treatment to the user (steps 320 and 322). In a further embodiment of the invention, apparatus 10 may provide a patient report that discloses the ECG data, the results of the cardiac marker test, any other patient parameter data, the suggestion of myocardial ischemia, and/or any suggested treatment. The patient report may be displayed on a visual display, such as visual display 202, or may be printed on a printer located within or remotely from apparatus 10.

Accordingly, there is provided apparatus and methods for documenting myocardial ischemia in patients. An apparatus is provided that is capable of documenting myocardial ischemia in a patient using, for example, electrocardial waveform data. The apparatus is further able to receive results of cardiac marker tests performed on the patient and to document the results. The apparatus then may provide the ECG data and the cardiac marker test results to the user of the apparatus or a physician, clinician or other technician for review and analysis so that the condition may be diagnosed and treated.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention. It being understood that various changes may be made in the function and arrangement of elements described in an exemplary embodiment without departing from the scope of the invention as set forth in the appended claims.

1. An apparatus for documenting the myocardial ischemia of a patient's heart, the apparatus comprising:
an ECG monitor and data collector configured to receive electrocardial data about the patient's heart;
a cardiac marker data collector configured to receive cardiac marker data about the patient's heart; and
a data processing and recording module in electrical communication with said ECG monitor and data collector and said cardiac marker data collector and configured to record said electrocardial data and said cardiac marker data.

2. The apparatus for documenting the myocardial ischemia of a patient's heart of claim 1, said data processing and recording module comprising at least one of a processor and a memory device.

3. The apparatus for documenting the myocardial ischemia of a patient's heart of claim 1, wherein the apparatus further comprises an user interface configured to permit entry of said cardiac marker data for receipt by said cardiac marker data collector.

4. The apparatus for documenting the myocardial ischemia of a patient's heart of claim 1, wherein said data processing and recording module is configured to diagnose myocardial ischemia based on said electrocardial data.

5. The apparatus for documenting the myocardial ischemia of a patient's heart of claim 4, wherein said data processing and recording module is configured to diagnose myocardial ischemia based on said electrocardial data and said cardiac marker data.

6. The apparatus for documenting the myocardial ischemia of a patient's heart of claim 5, wherein said data processing and recording module is configured to suggest a treatment for myocardial ischemia based on said electrocardial data and said cardiac marker data.

7. The apparatus for documenting the myocardial ischemia of a patient's heart of claim 1, the apparatus further comprising a display module in electrical communication with said data processing and recording module and configured to display at least one of said electrocardial data and said cardiac marker data.

8. The apparatus for documenting the myocardial ischemia of a patient's heart of claim 7, wherein said data processing and recording module is configured to suggest a treatment for myocardial ischemia based on said electrocardial data and said cardiac marker data and said display module is configured to display said suggested treatment.

9. The apparatus for documenting the myocardial ischemia of a patient's heart of claim 7, said display module comprising at least one of a visual display and a printer.

10. The apparatus for documenting the myocardial ischemia of a patient's heart of claim 1, the apparatus further comprising an interpretive ECG algorithm module in electrical communication with said data processing and recording module.

11. The apparatus for documenting the myocardial ischemia of a patient's heart of claim 1, wherein said data processing and recording module is configured to detect a change over time of said electrocardial data.

12. The apparatus for documenting the myocardial ischemia of a patient's heart of claim 1, wherein said data processing and recording module is configured to detect a change over time of said electrocardial data and said cardiac marker data.

13. The apparatus for documenting the myocardial ischemia of a patient's heart of claim 1, wherein said cardiac marker data collector is configured to identify a time and a date of receipt of said cardiac marker data.

14. The apparatus for documenting the myocardial ischemia of a patient's heart of claim 1, wherein said data processing and recording module is configured to generate a prompt to a user of the apparatus to perform a cardiac marker test.

15. The apparatus for documenting the myocardial ischemia of a patient's heart of claim 14, wherein said prompt comprises at least one of a visual signal and an auditory signal.

16. The apparatus for documenting the myocardial ischemia of a patient's heart of claim 1, wherein said data processing and recording module is configured to monitor a time period for performing a cardiac marker test on the patient and to generate a request for results of said cardiac marker test when said time period has expired.

17. The apparatus for documenting the myocardial ischemia of a patient's heart of claim 1, the apparatus further comprising a patient data collector that is in electrical communication with said data processing and recording module and is configured to receive patient data comprising at least one of a name of the patient, an identification number of the patient, an age of the patient, a sex of the patient, and a race of the patient.

18. The apparatus for documenting the myocardial ischemia of a patient's heart of claim 17, wherein the apparatus further comprises a user interface configured to permit entry of said patient data for receipt by said patient data collector.

19. The apparatus for documenting the myocardial ischemia of a patient's heart of claim 1, the apparatus further comprising at least one patient parameter monitor and collector that is in electrical communication with said data processing and recording module and that is configured to receive data regarding a physiological state of the patient.

20. The apparatus for documenting the myocardial ischemia of a patient's heart of claim 19, wherein said data regarding a physiological state of the patient comprises at least one of a heart rate of the patient, a blood pressure of the patient, a hemoglobin oxygen saturation of the patient, and an end-tidal carbon dioxide of the patient.

21. The apparatus for documenting the myocardial ischemia of a patient's heart of claim 1, the apparatus further comprising a housing enclosing said ECG monitor and data collector, said cardiac marker data collector and said data processing and recording module.

22. A method for documenting the myocardial ischemia of a patient's heart, the method comprising:
obtaining electrocardial data about the patient's heart;
receiving results of a cardiac marker test performed on the patient;
storing said electrocardial data and said results of said cardiac marker test in a patient report; and
displaying said patient report.

23. The method for documenting the myocardial ischemia of a patient's heart of claim 22, the step of displaying said patient report comprising displaying said patient report on a visual display.

24. The method for documenting the myocardial ischemia of a patient's heart of claim 22, the step of displaying said patient report comprising printing said patient report.

25. The method for documenting the myocardial ischemia of a patient's heart of claim 22, the method further comprising the step of identifying a time and a date of receipt of said cardiac marker data and storing said time and said date in said patient report.

26. The method for documenting the myocardial ischemia of a patient's heart of claim 22, the method further comprising the step of obtaining data regarding the physiological state of the patient.

27. The method for documenting the myocardial ischemia of a patient's heart of claim 26, the step of obtaining data regarding the physiological state of the patient comprising obtaining at least one of a heart rate of the patient, a blood pressure of the patient, a hemoglobin oxygen saturation of the patient and an end-tidal carbon dioxide of the patient.

28. The method for documenting the myocardial ischemia of a patient's heart of claim 26, the method further comprising the step of storing said data regarding the physiological state of the patient in said patient report.

29. The method for documenting the myocardial ischemia of a patient's heart of claim 22, the method further comprising the step of providing a prompt for the performance of a cardiac marker test.

30. The method for documenting the myocardial ischemia of a patient's heart of claim 22, the method further comprising the step of analyzing said electrocardial data to determine if myocardial ischemia is suggested by said electrocardial data.

31. The method for documenting the myocardial ischemia of a patient's heart of claim 30, the method further comprising the step of providing a notification that myocardial ischemia is diagnosed in the patient if said electrocardial data suggests myocardial ischemia.

32. The method for documenting the myocardial ischemia of a patient's heart of claim 30, the method further comprising the step of analyzing said electrocardial data to determine the severity of myocardial ischemia of the patient's heart.

33. The method for documenting the myocardial ischemia of a patient's heart of claim 30, the method further comprising the step of providing a prompt for the performance of a cardiac marker test if said electrocardial data suggests myocardial ischemia.

34. The method for documenting the myocardial ischemia of a patient's heart of claim 30, the method further comprising the step of analyzing said electrocardial data and said cardiac marker data to determine if myocardial ischemia is suggested by said electrocardial data and said cardiac marker data.

35. The method for documenting the myocardial ischemia of a patient's heart of claim 22, the method further comprising the step of analyzing said electrocardial data to detect a change over time of said electrocardial data.

36. The method for documenting the myocardial ischemia of a patient's heart of claim 22, the method further comprising the step of analyzing said electrocardial data and said cardiac marker data to detect a change over time of said electrocardial data and said cardiac marker data.

37. The method for documenting the myocardial ischemia of a patient's heart of claim 22, the method further comprising the steps of monitoring a time period for performing a cardiac marker test on the patient and generating a request for results of said cardiac marker test when said time period has expired.

38. The method for documenting the myocardial ischemia of a patient's heart of claim 22, the method further comprising the step of receiving patient data comprising at least one of a name of the patient, an identification number of the patient, an age of the patient, a sex of the patient, and a race of the patient.

39. The method for documenting the myocardial ischemia of a patient's heart of claim 38, the method further comprising the step of storing said patient data in said patient report.

40. An apparatus for documenting the myocardial ischemia in a patient's heart, the apparatus comprising:
means for receiving electrocardial data about the patient's heart;
means for receiving cardiac marker data about the patient's heart;
means for processing said electrocardial data and said cardiac marker data;
means for recording said electrocardial data and said cardiac marker data into a patient record; and
means for displaying said patient record.

41. The apparatus for documenting the myocardial ischemia of a patient's heart of claim 40, said means for processing said electrocardial data and said cardiac marker data comprising a processor.

42. The apparatus for documenting the myocardial ischemia of a patient's heart of claim 40, said means for recording said electrocardial data and said cardiac marker data comprising a memory device.

43. The apparatus for documenting the myocardial ischemia of a patient's heart of claim 40, wherein the apparatus further comprises means for permitting entry of said cardiac marker data for receipt by said cardiac marker data collector.

44. The apparatus for documenting the myocardial ischemia of a patient's heart of claim 43, wherein said means for permitting entry of said cardiac marker data comprises one of a keyboard, a keypad and a touch screen.

45. The apparatus for documenting the myocardial ischemia of a patient's heart of claim 40, wherein said means for processing said electrocardial data and said cardiac marker data further comprises means for diagnosing myocardial ischemia based on said electrocardial data.

46. The apparatus for documenting the myocardial ischemia of a patient's heart of claim 40, wherein said means for processing said electrocardial data and said cardiac marker data further comprises means for diagnosing myocardial ischemia based on said electrocardial data and said cardiac marker data.

47. The apparatus for documenting the myocardial ischemia of a patient's heart of claim 46, wherein said means for processing said electrocardial data and said cardiac marker data further comprises means for suggesting a treatment for myocardial ischemia.

48. The apparatus for documenting the myocardial ischemia of a patient's heart of claim 47, wherein said means for displaying said patient record further comprises means for displaying said suggested treatment.

49. The apparatus for documenting the myocardial ischemia of a patient's heart of claim 40, said means for displaying said patient record comprises at least one of a visual display and a printer.

50. The apparatus for documenting the myocardial ischemia of a patient's heart of claim 40, the apparatus further comprising means for interpreting said electrocardial data, said means for interpreting said electrocardial data in electrical communication with said means for processing said electrocardial data and said cardiac marker data.

51. The apparatus for documenting the myocardial ischemia of a patient's heart of claim 40, wherein said means for processing said electrocardial data and said cardiac marker data comprises means for detecting a change over time of said electrocardial data.

52. The apparatus for documenting the myocardial ischemia of a patient's heart of claim 40, wherein said means for processing said electrocardial data and said cardiac marker data comprises means for detecting a change over time of said electrocardial data and said cardiac marker data.

53. The apparatus for documenting the myocardial ischemia of a patient's heart of claim 40, wherein said means for receiving cardiac marker data comprises means for identifying a time and a date of receipt of said cardiac marker data.

54. The apparatus for documenting the myocardial ischemia of a patient's heart of claim 40, wherein said means for processing said electrocardial data and said cardiac marker data comprises means for generating a prompt to a user of the apparatus to perform a cardiac marker test.

55. The apparatus for documenting the myocardial ischemia of a patient's heart of claim 54, wherein said prompt comprises at least one of a visual signal and an auditory signal.

56. The apparatus for documenting the myocardial ischemia of a patient's heart of claim 40, wherein means for processing said electrocardial data and said cardiac marker data comprise means for monitoring a time period for performing a cardiac marker test on the patient and means for generating a request for results of said cardiac marker test when said time period has expired.

57. The apparatus for documenting the myocardial ischemia of a patient's heart of claim 40, the apparatus further comprising means for collecting patient data, wherein said means for collecting patient data is in electrical communication with said means for processing said electrocardial data and said cardiac marker data, and wherein said means for collecting patient data comprises means for receiving patient data comprising at least one of a name of the patient, an identification number of the patient, an age of the patient, a sex of the patient, and a race of the patient.

58. The apparatus for documenting the myocardial ischemia of a patient's heart of claim 57, wherein the apparatus further comprises means for permitting entry of said patient data for receipt by said means for collecting patient data.

59. The apparatus for documenting the myocardial ischemia of a patient's heart of claim 40, the apparatus further comprising at least one means for collecting patient parameter data, wherein said at least one means for collecting patient parameter data is in electrical communication with said means for processing said electrocardial data and said cardiac marker data, and wherein said at least one means for collecting patient parameter data comprises means for receiving data regarding a physiological state of the patient.

60. The apparatus for documenting the myocardial ischemia of a patient's heart of claim 59, wherein said data regarding a physiological state of the patient comprises at least one of a heart rate of the patient, a blood pressure of the patient, a hemoglobin oxygen saturation of the patient and an end-tidal carbon dioxide of the patient.

61. The apparatus for documenting the myocardial ischemia of a patient's heart of claim 40, the apparatus further comprising a housing enclosing at least a portion of said means for receiving electrocardial data about the patient's heart, said means for receiving cardiac marker data about the patient's heart and said means for processing said electrocardial data and said cardiac marker data.

62. A medical apparatus of the type that is configured to monitor the electrocardiogram waveform of a patient, the medical apparatus comprising:
a cardiac marker data collector configured to receive cardiac marker data about the patient's heart;
a data processor in electrical communication with said cardiac marker data collector;
a memory module in electrical communication with said data processor and configured to record said electrocardiogram waveform and said cardiac marker data; and
a display module in electrical communication with said data processor and configured to display at least one of the electrocardiogram waveform and said cardiac marker data.

63. The medical apparatus of claim 62, wherein the apparatus further comprises a user interface configured to permit entry of said cardiac marker data for receipt by said cardiac marker data collector.

64. The medical apparatus of claim 62, wherein said data processor is configured to diagnose myocardial ischemia based on the electrocardiogram waveform and said cardiac marker data.

65. The medical apparatus of claim 62, wherein said data processor is configured to diagnose myocardial ischemia based on the electrocardiogram waveform.

66. The medical apparatus of claim 64, wherein said data processor is configured to suggest a treatment for myocardial ischemia based on the electrocardiogram waveform and said cardiac marker data.

67. The medical apparatus of claim 66, wherein said display module is configured to display said suggested treatment.

68. The medical apparatus of claim 62, said display module comprising at least one of a visual display and a printer.

69. The medical apparatus of claim 62, wherein said data processor is configured to detect a change over time of said electrocardiogram waveform.

70. The medical apparatus of claim 62, wherein said data processor is configured to detect a change over time of said cardiac marker data.

71. The medical apparatus of claim 62, wherein said cardiac marker data collector is configured to identify a time and a date of receipt of said cardiac marker data.

72. The medical apparatus of claim 62, wherein said data processor is configured to generate a prompt to a user of the apparatus to perform a cardiac marker test.

73. The medical apparatus of claim 72, wherein said prompt comprises at least one of a visual signal and an auditory signal.

74. The medical apparatus of claim 62, wherein said data processor is configured to monitor a time period for performing a cardiac marker test on the patient and to generate a request for results of said cardiac marker test when said time period has expired.

75. The medical apparatus of claim 62, the medical apparatus further comprising a patient data collector that is in electrical communication with said data processor and is configured to receive patient data comprising at least one of a name of the patient, an identification number of the patient, an age of the patient, a sex of the patient, and a race of the patient.

76. The medical apparatus of claim 75, wherein the medical apparatus further comprises a user interface configured to permit entry of said patient data for receipt by said patient data collector.

77. The medical apparatus of claim 62, the apparatus further comprising at least one patient parameter monitor and collector that is in electrical communication with said data processor and that is configured to receive data regarding a physiological state of the patient.

78. The medical apparatus of claim 77, wherein said data regarding a physiological state of the patient comprises at least one of a heart rate of the patient, a blood pressure of the patient, a hemoglobin oxygen saturation of the patient and an end-tidal carbon dioxide of the patient.

79. The medical apparatus of claim 62, the apparatus further comprising a housing enclosing at least a portion of said cardiac marker data collector, at least a portion of said data processor and at least a portion of said memory module.
